(19) United States
(12) Patent Application Publication
(10) Pub. No.: US 2015/0107599 A1
(43) Pub. Date: Apr. 23, 2015

(54) POST VITRECTOMY POSITION STABILIZER

(71) Applicant: Saint Louis University, St. Louis, MO (US)

(72) Inventor: Michael T. Railey, St. Louis, MO (US)

(73) Assignee: Saint Louis University, St. Louis, MO (US)

(21) Appl. No.: 14/057,617

(22) Filed: Oct. 18, 2013

(51) Int. Cl. A61F 5/37

(52) U.S. Cl. CPC A61F 5/3707
USPC 128/845

(57) ABSTRACT

Post vitrectomy head stabilization devices are disclosed. The post vitrectomy head stabilization devices include a head strap connected to a head support rod, a chin strap connected to a chin support rod and a torso belt connected to the head support rod and connected to the chin support rod. The post vitrectomy head stabilization devices of the present disclosure advantageously allow a user to maintain a face down head position while the user's torso adopts an upright position such as when the user is standing, walking or seated.

### BACKGROUND

The present disclosure relates generally to medical devices. More particularly, the present disclosure is directed to devices for post vitrectomy head positioning.

Vitrectomy is a surgical procedure involving the removal of the vitreous gel from the middle of the eye. Vitrectomy may be done when there is a retinal detachment or any retinal repair such as macular holes, macular pucker, vitreous detachment, eye floaters and diabetic retinopathy, because removing the vitreous gel allows for better access to the back of the eye. Vitreous gel may also be removed if blood in the vitreous gel (vitreous hemorrhage) does not clear on its own.

At the end of the surgery, silicone oil or a gas is injected into the eye to replace the vitreous gel and restore normal pressure in the eye. Following surgery, the patient may need to maintain head position in a certain way such that the oil or gas bubble can push against the detachment. Typically, positioning requires that the patient lie or sit face down. Face down positioning begins immediately after the operation and continues for a period of seven or more days.

Remaining face down is important because the gas bubble can move to the anterior (top) part of the eye when the head is upright which can leave the retinal tear or detachment exposed to further pulling and irritation from the vitreous gel and eye fluids, preventing healing. Anterior movement of the air bubble within the eye can also accelerate cataract formation, raise intraocular pressure and damage the cornea. The success rate of vitrectomy in repairing the eye and restoring vision is near 90% when patients using proper vitrectomy recovery equipment are able to stay face down during the entire recovery period.

Equipment is available to support sitting and lying face down head positioning. For example, recovery chairs are available that allow patients a number of sitting positions. Recovery chairs typically include a padded horseshoe-shaped face cradle or headrest on which the patient's forehead and cheeks contact for support while the patient's face is positioned in the open space to allow unobstructed breathing and communicating. Recovery chairs may also include a chest plate on which the patient can lean for upper body support. The recovery chairs also include a seat and leg-rests that allow for different foot positioning. Recovery devices are also available for providing face down sleep support. Face down sleep support devices typically include a padded horseshoe-shaped face cradle or headrest on which the patient's forehead and cheeks contact for support while the patient's face is positioned in the open space.

Compliance with the face down positioning after vitrectomy can be the most difficult post-operative requirements. While post-vitrectomy devices are available for supporting face down positioning while sitting and sleeping, these devices only allow for limited or no mobility.

Accordingly, there is a need for alternative post-vitrectomy devices for supporting face down positioning, but that also allows for other patient positioning such as when the patient is standing or seated, as well as allows for patient mobility such as walking.

### BRIEF DESCRIPTION

In one aspect, the present disclosure is directed to a post vitrectomy head stabilization device for supporting a face down head position of a user comprising a head strap 12 connected to a head support rod 14, a chin strap 16 connected to a chin support rod 18 and a torso belt 20 connected to the head support rod 14 and connected to the chin support rod 18.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a photograph of an exemplary post vitrectomy head stabilization device for supporting a face down head position of a user.

FIG. 2 is a side view schematic illustration of the post vitrectomy head stabilization device worn by a user.

FIG. 3 is a front-on view schematic illustration of the post vitrectomy head stabilization device worn by a user.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

In accordance with the present disclosure, post vitrectomy head stabilization devices for supporting a face down head position of a user are described. The post vitrectomy head stabilization devices advantageously allow a user to maintain a face down head position while the user's torso adopts an upright position such as when the user is standing, walking or seated. As used herein, “user” refers to a patient who has undergone an eye surgery such as, for example, vitrectomy in which silicone oil or a gas is injected into the eye such that the user may need to maintain her/his head position in a certain way such that the oil or gas bubble remains properly positioned.

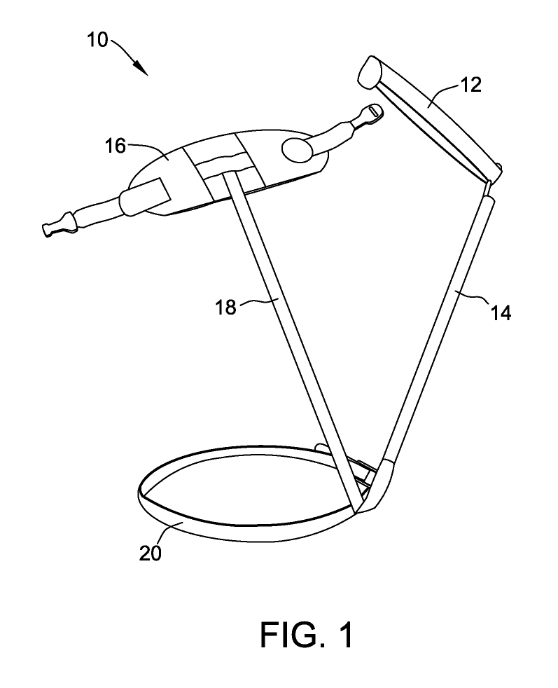

FIG. 1 is photograph of an exemplary embodiment of a post vitrectomy head stabilization device indicated generally at 10 including a head strap 12, a head support rod 14, a chin strap 16, a chin support rod 18 and a torso belt 20. The head support rod 14 is connected to the head strap 12 at one end and connected to a torso belt 20 at the other end. The chin support rod 18 is connected to the chin strap 16 at one end and connected to the torso belt 20 at the other end. The chin support rod 18 is connected to the torso belt 20 at a region proximate to a region where the head support rod 14 is connected to the torso belt 20. The mechanisms for connected the chin support rod 18 to the chin strap 16 and the head support rod 14 to the head strap 12 and for connected the chin support rod 18 and the head support rod 14 to the torso belt 20 should be fashioned such that they securely maintain the connection while pressure is applied to the chin support rod 18 and the head support rod 14 when worn by the user to support the face down head position. The chin support rod 18, the head support rod 14 and the torso belt 20 can be coupled (or connected) in any manner known to those skilled in the art. For example, the chin support rod 18 and the head support rod 14 can each be connected to the torso belt 20 using pivot mounts, which allow some movement at the connection point. In addition to, or alternatively, the chin support rod 18 can be connected to the chin strap 16 and the head support rod 14 can be connected to the head strap 12 in any manner known to those skilled in the art. For example, the chin support rod 18 can be connected to the chin strap 16 and the head support rod 14 can be connected to the head strap 12 using pivot mounts, which allow some movement at the connection point. In addition to, or alternatively, the chin support rod 18 can be connected to the chin strap 16 and the head support rod 14 can be connected to the head strap 12 in a manner that allows for little or no movement at the connection point.

The head strap 12 is positioned to wrap around the head from the forehead, extending past the temple regions of the head to the back of the head. In one embodiment, the head strap 12 can be a single fit (i.e., not adjustable). To provide a comfortable and stable fit of the head strap 12 on the user's head, a fixed size head strap 12 of the desired size can be selected by one skilled in the art such as a medical professional, for example. In another embodiment, the head strap 12 can be adjustable as to the user's head size to provide a comfortable and stable fit of the head strap 12 on the user's head. The head strap 12 can be adjustable by using, for example, a buckle, a hook-and-loop fastener (i.e., VELCRO®), snaps, buttons and other fasteners known to those skilled in the art. Two ends of the head strap 12 can be brought together such that the ends overlap and are held in place by the fastener. The head strap 12 can also be adjustable by using, for example, an elastic material, that can stretch to fit the user's head. To enhance a user's comfort, the head strap 12 can be made from a lightweight material. The head strap 12 can also be made from a material that can wick perspiration and/or absorb perspiration such as terrycloth, for example.

At the point where the end of the head support rod 14 is coupled to the head strap 12, the head strap 12 can have a forehead rest 22. The forehead rest 22 forms part of the head strap 12. The forehead rest 22 can be made as part of the head strap 12 and or be made separately from the head strap 12 and fastened to the head strap 12 when the device 10 is worn by the user. In another embodiment, the forehead rest 22 can also be made as part of the head support rod 14. In another embodiment, the forehead rest 22 can be made separately from the head support rod 14 and coupled to the end of the head support rod 14 when the device 10 is worn by the user. To enhance a user's comfort, the forehead rest 22 can be made from a lightweight-cushioned (or padded) material. The forehead rest 22 can also have a wider width than the remainder of the head strap 12 to provide comfort and additional surface area for the user's forehead to contact the forehead rest 22 and distribute pressure experienced on the user's forehead when the user's head is positioned in contact with the forehead rest 22. This provides a cushion for the forehead when the device 10 is worn and the user's head is in a face down position and pressure is being applied to the head support rod 14.

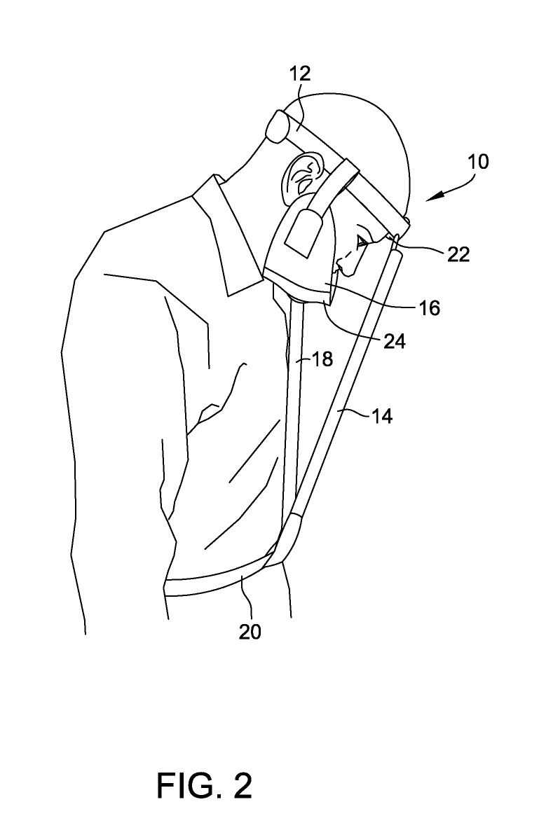

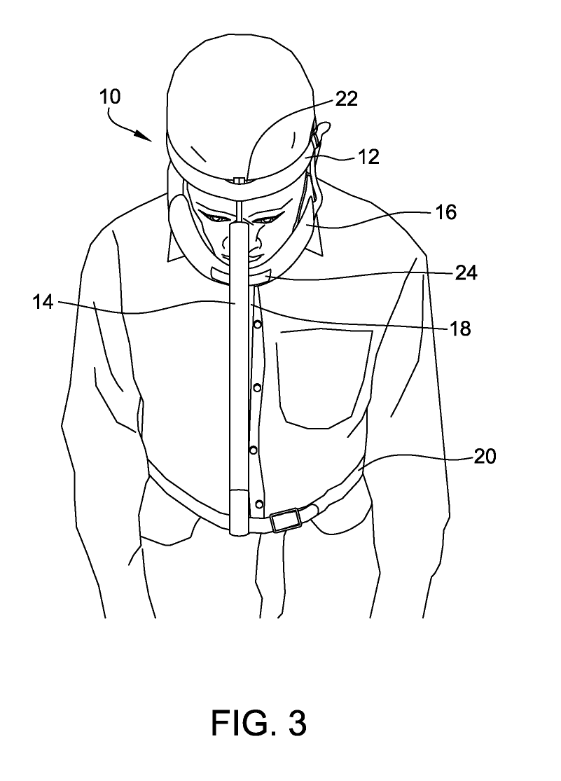

In one embodiment, the chin strap 16 can be a single fit (i.e., not adjustable). To provide a comfortable and stable fit of the chin strap 16 on the user's head, a fixed size chin strap 16 of the desired size can be selected by one skilled in the art such as a medical professional, for example. In another embodiment, the chin strap 16 can be adjustable as to the user's head size to provide a comfortable and stable fit of the chin strap 16 on the user's head. The chin strap 16 can be adjustable by using, for example, a buckle, a hook-and-loop fastener (i.e., VELCRO®), snaps, buttons and other fasteners known to those skilled in the art. Two ends of the chin strap 16 can be brought together such that the ends overlap and are held in place by the fastener. The chin strap 16 can also be adjustable by using, for example, an elastic material, that can stretch to fit the user's head. In one embodiment, the chin strap 16 can wrap around (and over) the user's head. In another embodiment as shown in FIGS. 2 and 3, the chin strap 16 can be connected to the head strap 12 and not extend over the user's head. The chin strap 16 can be connected to the head strap 12 using a fastener such as, for example, a buckle, a hook-and-loop fastener (i.e., VELCRO®), snaps, buttons, thread (i.e., sewn) and other fasteners known to those skilled in the art. To enhance a user's comfort, the chin strap 16 can be made from a lightweight material. The chin strap 16 can also be made from a material that can wick perspiration and/or absorb perspiration such as terrycloth, for example.

At the point where the end of the chin support rod 18 is coupled to the chin strap 16, the chin strap 16 can have a chin rest 24. The chin rest 24 can form part of the chin strap 16. To enhance a user's comfort, the chin rest 24 can be can be made from a lightweight-cushioned (or padded) material. The chin rest 24 can also have a wider width than the remainder of the chin strap 16 to provide comfort and additional surface area for the user's chin to contact the chin rest 24 and distribute pressure experienced by the user's chin when the user's chin is positioned in contact with the chin rest 24. This provides a cushion for the user's chin when the device 10 is worn and the user's head is in a face down position and pressure is being applied to the chin support rod 18.

The torso belt 20 can be adjustable as to the user's torso size to provide a comfortable and stable fit of the torso belt 20 around the user's torso. The torso belt 20 can be a single fit or be adjustable. The torso belt 20 can be adjustable by using, for example, a buckle a hook-and-loop fastener (i.e., VELCRO®), snaps, buttons and other fasteners known to those skilled in the art. To enhance a user's comfort, the torso belt 20 can be made from a lightweight material. Two ends of the torso belt 20 can be brought together such that the ends overlap and are held in place by the fastener. The torso belt 20 can also be made from a cushioned (or padded) material to enhance a user's comfort. In use, the torso belt 20 can be positioned around the user's waist, above the user's waist, or below the user's waist. Preferably, the torso belt 20 can be positioned around the user's waist such that the bones of the hip prevent the torso belt 20 from sliding too low such that the user's head position drops past the desired face down position. In one embodiment, the torso belt 20 can have a width that allows the user to thread a portion or the entire torso belt 20 through the belt loops of the user's pants or trousers. In this embodiment, threading the torso belt 20 through the belt loops prevents the torso belt 20 from sliding too low such that the user's head position drops past the desired face down position. This embodiment may also provide feedback to the user with regard to how much pressure the user is placing on the head support rod 14 and/or chin support rod 18. For example, if the user places too much pressure on the head support rod 14 and/or chin support rod 18 such that the user's head position drops past the desired face down position, the torso belt 20 will “pull” on the user's pants or trousers, which provides an indication that the user is applying too much downward pressure and may no longer be maintaining the proper face down position.

In the exemplary embodiment shown in FIGS. 1-3, the head support rod 14 and the chin support rod 18 are two separate rods; one rod providing the head support rod 14 and a second rod providing the chin support rod 18. In another embodiment, a forked or “Y” shaped rod can be used. In the forked or “Y” shaped rod embodiment, a single portion is coupled to the torso belt 20. As the rod extends up toward the user's head, the rod forks such that one rod extends toward the user's forehead to provide a head support rod 14 and a second rod extends toward the user's chin to form a chin support rod 18.

The material used to make the head support rod 14 and the chin support rod 18 can be a semi-flexible material or a rigid material. Suitable materials can be, for example, metals, plastics, carbon fiber, wood and combinations thereof. The material can also be lightweight material to provide comfort for the user.

In use, the head support rod 14 can be a length that is longer than the length of the chin support rod 18. In one embodiment, the length of the head support rod 14 and the chin support rod 18 can be fixed (i.e., a single length that is not adjustable). In another embodiment, the length of the head support rod 14 and the chin support rod 18 can be adjustable. For example, the adjustable length head support rod 14 and the adjustable chin support rod 18 can be adjusted in length by a telescoping method in which two or more sections having different diameters are assembled in which sections can be inserted within one another to form a segmented rod. A portion of the sections can be withdrawn and retracted until the desired lengths of the head support rod 14 and the chin support rod 18 are achieved. For embodiments having an adjustable head support rod 14 and/or an adjustable chin support rod 18, fasteners can be used to hold segmented shorter sections in place and prevent the segment from moving once a desired length is achieved. The fasteners can be, for example, screws, bolts, screw knobs, pin and hole, quick-release, flip-lock and other fasteners known by those skilled in the art. In another embodiment, a fixed length head support rod 14 can be used together with an adjustable length chin support rod 18. In yet another embodiment, a fixed length chin support rod 18 can be used together with an adjustable length head support rod 14. As understood by one skilled in the art such as, for example, a medical professional, the lengths of the head support rod 14 and the chin support rod 18 will vary depending on the user. Thus, the lengths of the head support rod 14 and the chin support rod 18 will be determined for a specific user based on a fitting such that the desired face down head position can be achieved.

As illustrated in FIGS. 2 and 3, when worn by the user, the head support rod 14 is positioned adjacent the user's forehead. Downward pressure exerted by the user on the head support rod 14 becomes transferred to the connection of the head support rod 14 to the torso belt 20 to support the weight of the user's head when in the face down position. Downward pressure exerted by the user on the chin support rod 18 becomes transferred to the connection of the chin support rod 18 to the torso belt 20 to support the weight of the user's head when in the face down position. In combination, the downward pressures exerted by the user on the head support rod 14 and the chin support rod 18 advantageously allows the user to maintain the face down head positioning while the user's torso is in an upright (vertical to the floor) position such as when the user is standing, walking or seated. This is in contrast to the user's body positioning when lying down in which the user's body is horizontal to the floor.

The post vitrectomy head stabilization devices can further include attachments such as, for example, a mirror. A mirror allows a user to view the area in front of the user while maintaining the face down head position. A mirror can also allow a user to watch television, view a computer monitor and perform other activities where the user may wish to view the area in front of her/him while maintaining the face down head position.

The post vitrectomy head stabilization devices can further include attachments such as, for example, a level. A level provides the user feedback regarding correct face down head positioning. Any suitable level known to those skilled in the art may be used. Suitable levels can be, for example, a bubble level (or spirit level) such as, for example, a tubular spirit level and a bulls-eye spirit level. The level may be attached to the chin support rod 18 using any fastening system known to those skilled in the art. The level may also be attached to the head support rod 14 using any fastening system known to those skilled in the art.

The post vitrectomy head stabilization devices of the present disclosure advantageously allow a user to maintain a face down head position while the user's torso adopts an upright position such as when the user is standing or seated. Additionally, the post vitrectomy head stabilization devices allow a user to adopt additional positions while recovering from vitrectomy and maintain a face down head position previously allowed only by the user lying horizontal to the floor or sitting in specially adapted chairs that permit the user to lean forward against a chest rest. The post vitrectomy head stabilization devices of the present disclosure allow a user to now adopt standing and sitting positions in addition to the forward leaning seated position and horizontal lying position while recovering. The ability of a user to now adopt standing and seated positions, as well as allowing the user to walk, are likely to aid the user in complying with face down head positioning during the recovery phase, and thus, lead to better post-surgical outcomes.

In view of the above, it will be seen that the several advantages of the disclosure are achieved and other advantageous results attained. As various changes could be made in the above devices without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A post vitrectomy head stabilization device for supporting a face down head position of a user comprising: a head strap connected to a head support rod, a chin strap connected to a chin support rod and a torso belt connected to the head support rod and connected to the chin support rod.

2. The post vitrectomy head stabilization device of claim 1, wherein the chin strap is connected to the head strap.

3. The post vitrectomy head stabilization device of claim 1, further comprising a forehead rest.

4. The post vitrectomy head stabilization device of claim 1, further comprising a chin rest.

5. The post vitrectomy head stabilization device of claim 1, wherein the head support rod length is adjustable.

6. The post vitrectomy head stabilization device of claim 5, wherein the head support rod length is adjustable by telescoping.

7. The post vitrectomy head stabilization device of claim 1, wherein the chin support rod length is adjustable.

8. The post vitrectomy head stabilization device of claim 7, wherein the chin support rod length is adjustable by telescoping.

9. The post vitrectomy head stabilization device of claim 1, wherein the head strap is adjustable.

10. The post vitrectomy head stabilization device of claim 1, wherein the head strap is cushioned.

11. The post vitrectomy head stabilization device of claim 3, wherein the forehead rest is cushioned.

12. The post vitrectomy head stabilization device of claim 1, wherein the chin strap is adjustable.

13. The post vitrectomy head stabilization device of claim 1, wherein the chin strap is cushioned.

14. The post vitrectomy head stabilization device of claim 4, wherein the chin rest is cushioned.

15. The post vitrectomy head stabilization device of claim 1, further comprising an attachment.

16. The post vitrectomy head stabilization device of claim 15, wherein the attachment comprises a mirror.

17. The post vitrectomy head stabilization device of claim 15, wherein the attachment comprises a level.

18. The post vitrectomy head stabilization device of claim 17, wherein the level is selected from the group consisting of a tubular spirit level and a bulls-eye spirit level.
